(19) United States
(12) Patent Application Publication
(10) Pub. No.: US 2023/0398349 A1
(43) Pub. Date: Dec. 14, 2023

(54) ESTIMATION OF ELECTROPORATION PARAMETER LEVELS

(71) Applicant: Cochlear Limited, Macquarie University, NSW (AU)

(72) Inventors: Paul Michael Carter, Macquarie University (AU); Daniel Smyth, Mechelen (BE); Wolfram Frederik Dueck, Hannover (DE); Stefan Jozef Mauger, East Melbourne (AU)

(21) Appl. No.: 18/250,340

(22) PCT Filed: Oct. 15, 2021
(86) PCT No.: PCT/IB2021/059539

§ 371 Date: Apr. 24, 2023

Related U.S. Application Data
(60) Provisional application No. 63/117,266, filed on Nov. 23, 2020.

(51) Int. Cl. A61N 1/32; A61N 1/36; A61N 1/05; A61B 5/0536; A61B 5/388; A61B 5/00

(52) U.S. Cl. CPC A61N 1/327; A61N 1/36038; A61N 1/0541; A61B 5/0536; A61B 5/388; A61B 5/686; A61B 5/0071

(57) ABSTRACT

A method includes receiving information regarding a position and/or an orientation of an implantable device relative to a body portion of a recipient during and/or after implantation of at least a portion of the device on and/or into the body portion. The device includes a plurality of electrodes configured to apply electroporation to first cells of the body portion while not damaging second cells of the body portion. The method includes estimating, in response at least in part to the information, first interactions of the electrodes with respect to the first cells. The method further includes estimating, in response at least in part to the information, second interactions of the electrodes with respect to the second cells. The method further includes generating, in response at least in part to the estimated first interactions and the estimated second interactions, values of electrical parameters configured to be provided to the plurality of electrodes to electroporate to the first cells while not damaging the second cells.

### BACKGROUND

### Field

The present application relates generally to systems and methods for applying electroporation to cells within the body of a recipient, and more specifically, to facilitating setting electroporation signal levels.

### Description of the Related Art

Medical devices have provided a wide range of therapeutic benefits to recipients over recent decades. Medical devices can include internal or implantable components/devices, external or wearable components/devices, or combinations thereof (e.g., a device having an external component communicating with an implantable component). Medical devices, such as traditional hearing aids, partially or fully-implantable hearing prostheses (e.g., bone conduction devices, mechanical stimulators, cochlear implants, etc.), pacemakers, defibrillators, functional electrical stimulation devices, and other medical devices, have been successful in performing lifesaving and/or lifestyle enhancement functions and/or recipient monitoring for a number of years.

The types of medical devices and the ranges of functions performed thereby have increased over the years. For example, many medical devices, sometimes referred to as “implantable medical devices,” now often include one or more instruments, apparatus, sensors, processors, controllers or other functional mechanical or electrical components that are permanently or temporarily implanted in a recipient. These functional devices are typically used to diagnose, prevent, monitor, treat, or manage a disease/injury or symptom thereof, or to investigate, replace or modify the anatomy or a physiological process. Many of these functional devices utilize power and/or data received from external devices that are part of, or operate in conjunction with, implantable components.

### SUMMARY

In one aspect disclosed herein, a method comprises receiving information regarding a position and/or an orientation of an implantable device relative to a body portion of a recipient during and/or after implantation of at least a portion of the device on and/or into the body portion. The device comprises a plurality of electrodes configured to apply electroporation to first cells of the body portion while not damaging second cells of the body portion. The method comprises estimating, in response at least in part to the information, first interactions of the electrodes with respect to the first cells. The method further comprises estimating, in response at least in part to the information, second interactions of the electrodes with respect to the second cells. The method further comprises generating, in response at least in part to the estimated first interactions and the estimated second interactions, values of electrical parameters configured to be provided to the plurality of electrodes to electroporate to the first cells while not damaging the second cells.

In another aspect disclosed herein, a system comprises at least one data input interface configured to receive data indicative of a pose of a medical device relative to a body portion of a recipient. The medical device comprises a plurality of electrodes configured to apply electroporation electrical signals to the body portion. The system further comprises at least one controller in operative communication with the at least one data input interface. The at least one controller is configured to access anatomical model data of the body portion. The at least one controller is further configured to, in response at least in part to the anatomical model data and the data indicative of the pose of the medical device, generate estimated first electrical parameter values at a target region of the body portion and estimated second electrical parameter values at a non-target region of the body portion resulting from electrical signals applied to the plurality of electrodes. The at least one controller is further configured to generate an evaluation of whether the estimated first electrical parameter values are sufficient for electroporating the target region and/or the estimated second electrical parameter values are insufficient for damaging the non-target region. The system further comprises at least one output interface in operative communication with the at least one controller, the at least one output interface configured to provide information indicative of the evaluation.

In another aspect disclosed herein, a non-transitory computer readable storage medium has stored thereon a computer program that instructs a computer system to provide real-time information regarding electroporation to be applied by electrodes of a device to first cells of a body portion of a recipient during and/or after implantation of the device on and/or within the body portion by at least: receiving information regarding a pose of the device relative to the body portion during and/or after implantation of the device; estimating, in response at least in part to the information, first interactions of the electrodes with respect to the first cells; estimating, in response at least in part to the information, second interactions of the electrodes with respect to second cells different from the first cells; and generating, in response at least in part to the estimated first interactions and the estimated second interactions, values of electrical parameters configured to be provided to the plurality of electrodes to electroporate to the first cells while not damaging the second cells.

### BRIEF DESCRIPTION OF THE DRAWINGS

Implementations are described herein in conjunction with the accompanying drawings, in which:

FIG. 1 is a perspective view of an example auditory prosthesis implanted in a recipient with a stimulation assembly inserted into the cochlea in accordance with certain implementations described herein;

FIG. 2 is cross-sectional view of the cochlea illustrating the stimulating assembly partially implanted therein in accordance with certain implementations described herein;

FIGS. 3A-3C schematically illustrate examples of a system in accordance with certain implementations described herein; and

FIGS. 4A and 4B are flow diagram of examples of a method in accordance with certain implementations described herein.

### DETAILED DESCRIPTION

Certain implementations described herein provide a system and method for optimizing electroporation using implantable electrodes. By estimating the electrical interactions between the electrodes and the cells to be electroporated and other cells to not be electroporated (e.g., using data generated using the electrodes and/or imaging data) and, in response, determining the electroporation parameter (e.g., electrical voltage; electrical current; electrical field; electrical field gradient; electrical charge) values to be applied to the electrodes, certain implementations described herein achieve the correct stimulus dose (e.g., to make the whole cochlea cell continuum more equally permeable; to reduce the amount of non-target tissue that is electroporated).

The teachings detailed herein are applicable, in at least some implementations, to any type of implantable medical device (e.g., implantable stimulation system) configured to apply electroporation signals (e.g., electrical voltages; electrical currents; electrical fields; electrical field gradient; electrical charges) to cells on or within the recipient's body. The implantable medical device can be configured to deliver only electroporation signals or can be configured to deliver stimulation signals in addition to the electroporation signals. For example, the implantable medical device can comprise a first portion implanted on or within the recipient's body and configured to provide electroporation signals or both stimulation signals and electroporation signals to a portion of the recipient's body and a second portion (e.g., implanted on or within the recipient or external to the recipient's body) configured to provide control signals to the first portion. The implantable medical device can comprise an auditory prosthesis system utilizing an implantable actuator assembly that generates electrical, magnetic, and/or optical stimulation signals to the recipient that are perceived by the recipient as sounds. Examples of auditory prosthesis systems compatible with certain implementations described herein include but are not limited to: electro-acoustic electrical/acoustic systems, cochlear implant devices, electro-acoustic implant devices, auditory brainstem implant (ABI) devices, auditory midbrain implant (AMI) devices, or other types of auditory prosthesis devices, and/or combinations or variations thereof, or any other suitable hearing prosthesis system with or without one or more external components. Implementations can include any type of medical device that can utilize the teachings detailed herein and/or variations thereof.

Merely for ease of description, apparatus and methods disclosed herein are primarily described with reference to an illustrative medical device, namely a cochlear implant. However, the teachings detailed herein and/or variations thereof may also be used with a variety of other medical devices that provide a wide range of therapeutic benefits to recipients, patients, or other users. For example, the techniques presented herein may be used with other hearing prostheses, including acoustic hearing aids, bone conduction devices, middle ear auditory prostheses, direct acoustic stimulators, other electrically simulating auditory prostheses (e.g., auditory brain stimulators), etc. In some implementations, the teachings detailed herein and/or variations thereof can be utilized in other types of implantable medical devices beyond auditory prostheses. For example, apparatus and methods disclosed herein and/or variations thereof may also be used with one or more of the following: vestibular devices (e.g., vestibular implants); visual devices (e.g., bionic eyes); visual prostheses (e.g., retinal implants); sensors; cardiac pacemakers; drug delivery systems; defibrillators; functional electrical stimulation devices; catheters; neurostimulation devices (e.g., brain stimulation implants; seizure devices; devices for monitoring and/or treating epileptic events; deep brain stimulation devices); sleep apnea devices; electroporation; pain relief devices; swallowing treatment devices (e.g., devices for treating difficulties with the hyoglossus and/or thyrohyoid muscles); dysphagia treatment devices; devices for treating dry mouth (e.g., xerostomia or hyposalivation), intra-muscular devices (e.g., for treating excessive or absence of muscle movement due to stroke, Parkinson's disease, or other brain disorders), devices for treating hypertension (e.g., by stimulating the carotid sinus barosensory system); devices for treating the gastrointestinal tract; devices for treating or removing cancerous tissue; etc.

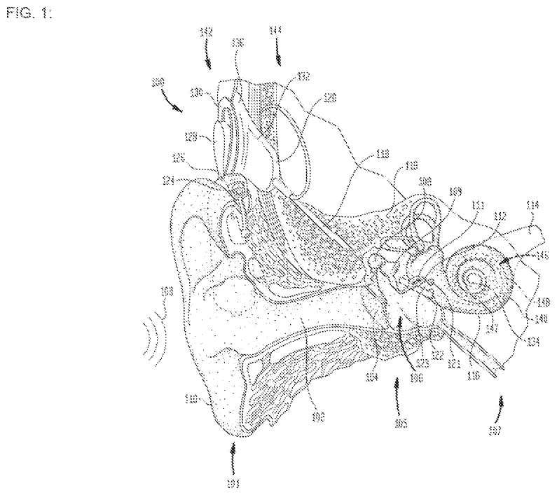

FIG. 1 is a perspective view of an example auditory prosthesis 100 (e.g., cochlear implant), implanted in a recipient with a stimulation assembly 118 inserted into the cochlea 140 in accordance with certain implementations described herein. As shown in FIG. 1, the recipient has an outer ear 101, a middle ear 105, and an inner ear 107. In a fully functional ear, the outer ear 101 comprises an auricle 110 and an ear canal 102. An acoustic pressure or sound wave 103 is collected by the auricle 110 and is channeled into and through the ear canal 102. Disposed across the distal end of the ear canal 102 is a tympanic membrane 104 which vibrates in response to the sound wave 103. This vibration is coupled to oval window or fenestra ovalis 112 through three bones of middle ear 105, collectively referred to as the ossicles 106 and comprising the malleus 108, the incus 109, and the stapes 111. The bones 108, 109, and 111 of the middle ear 105 serve to filter and amplify the sound wave 103, causing the oval window 112 to articulate, or vibrate in response to vibration of the tympanic membrane 104. This vibration sets up waves of fluid motion of the perilymph within the cochlea 140. Such fluid motion, in turn, activates tiny hair cells (not shown) inside the cochlea 140. Activation of the hair cells causes appropriate nerve impulses to be generated and transferred through the spiral ganglion cells (not shown) and auditory nerve 114 to the brain (also not shown) where they are perceived as sound.

As shown in FIG. 1, the example auditory prosthesis 100 comprises one or more components which are temporarily or permanently implanted in the recipient. The example auditory prosthesis 100 is shown in FIG. 1 with an external component 142 which is directly or indirectly attached to the recipient's body, and an internal component 144 which is temporarily or permanently implanted in the recipient (e.g., positioned in a recess of the temporal bone adjacent to the auricle 110 of the recipient). The external component 142 typically comprises one or more sound input elements (e.g., an external microphone 124) for detecting sound, a sound processing unit 126 (e.g., disposed in a Behind-The-Ear unit), a power source (not shown), and an external transmitter unit 128. In the illustrative implementation of FIG. 1, the external transmitter unit 128 comprises an external coil 130 (e.g., a wire antenna coil comprising multiple turns of electrically insulated single-strand or multi-strand platinum or gold wire) and, preferably, a magnet (not shown) secured directly or indirectly to the external coil 130. The external coil 130 of the external transmitter unit 128 is part of an inductive radio frequency (RF) communication link with the internal component 144. The sound processing unit 126 processes the output of the microphone 124 that is positioned externally to the recipient's body, in the depicted implementation, by the recipient's auricle 110. The sound processing unit 126 generates encoded signals, sometimes referred to herein as encoded data signals, which are provided to the external transmitter unit 128 (e.g., via a cable).

The power source of the external component 142 is configured to provide power to the auditory prosthesis 100, where the auditory prosthesis 100 includes a battery (e.g., located in the internal component 144, or disposed in a separate implanted location) that is recharged by the power provided from the external component 142 (e.g., via a transcutaneous energy transfer link). The transcutaneous energy transfer link is used to transfer power and/or data to the internal component 144 of the auditory prosthesis 100. Various types of energy transfer, such as infrared (IR), electromagnetic, capacitive, and inductive transfer, may be used to transfer the power and/or data from the external component 142 to the internal component 144. During operation of the auditory prosthesis 100, the power stored by the rechargeable battery is distributed to the various other implanted components as needed.

The internal component 144 comprises an internal receiver unit 132, a stimulator unit 120, and an elongate stimulation assembly 118. In some implementations, the internal receiver unit 132 and the stimulator unit 120 are hermetically sealed within a biocompatible housing, sometimes collectively referred to as a stimulator/receiver unit. The internal receiver unit 132 comprises an internal coil 136 (e.g., a wire antenna coil comprising multiple turns of electrically insulated single-strand or multi-strand platinum or gold wire), and preferably, a magnet (also not shown) fixed relative to the internal coil 136. The internal coil 136 receives power and/or data signals from the external coil 130 via a transcutaneous energy transfer link (e.g., an inductive RF link). The stimulator unit 120 generates electrical stimulation signals based on the data signals, and the stimulation signals are delivered to the recipient via the elongate stimulation assembly 118.

The elongate stimulation assembly 118 has a proximal end connected to the stimulator unit 120, and a distal end implanted in the cochlea 140. The stimulation assembly 118 extends from the stimulator unit 120 to the cochlea 140 through the mastoid bone 119. In some implementations, the stimulation assembly 118 may be implanted at least in the basal region 116, and sometimes further. For example, the stimulation assembly 118 may extend towards the apical end of the cochlea 140, referred to as the cochlea apex 134. In certain circumstances, the stimulation assembly 118 may be inserted into the cochlea 140 via a cochleostomy 122. In other circumstances, a cochleostomy 122 may be formed through the round window 121, the oval window 112, the promontory 123, or through an apical turn 147 of the cochlea 140.

The elongate stimulation assembly 118 comprises a longitudinally aligned and distally extending array 146 (e.g., electrode array; contact array) of stimulation elements 148 (e.g., electrical electrodes; electrical contacts; optical emitters; optical contacts). For example, the stimulation elements 148 can comprise intra-cochlear electrodes (ICEs) and/or extra-cochlear electrodes (ECEs). The stimulation elements 148 are longitudinally spaced from one another along a length of the elongate body of the stimulation assembly 118. For example, the stimulating assembly 118 can comprise an array 146 comprising twenty-two (22) stimulation elements 148 that are configured to deliver stimulation to the cochlea 140. Although the array 146 of stimulation elements 148 can be disposed on the stimulation assembly 118, in most practical applications, the array 146 is integrated into the stimulation assembly 118 (e.g., the stimulation elements 148 of the array 146 are disposed in the stimulation assembly 118). As noted, the stimulator unit 120 generates stimulation signals (e.g., electrical signals; optical signals) which are applied by the stimulation elements 148 to the cochlea 140, thereby stimulating the auditory nerve 114.

A variety of types of intra-cochlear stimulation assemblies 118 are compatible with certain embodiments described herein, including but not limited to: short, straight, and perimodiolar. A perimodiolar stimulation assembly 118 is configured to adopt a curved configuration during and/or after implantation into the cochlea 140. To achieve this, in certain implementations, the perimodiolar stimulation assembly 118 is pre-curved to the same general curvature of the cochlea 140. Such examples of the stimulation assembly 118 can be held straight by, for example, a stiffening stylet (not shown) or sheath which is removed during implantation, or alternatively varying material combinations or the use of shape memory materials, so that the stimulation assembly 118 may adopt its curved configuration when in the cochlea 140. Other methods of implantation, as well as other stimulation assemblies 118 which adopt a curved configuration, may be used. The stimulation assembly 118 of certain other implementations comprises a non-perimodiolar stimulation assembly 118. For example, the stimulation assembly 118 can comprise a straight stimulation assembly 118 or a mid-scala assembly which assumes a mid-scala position during or following implantation. Alternatively, the stimulation assembly 118 can comprise a short electrode implanted into at least the basal region of the cochlea 140.

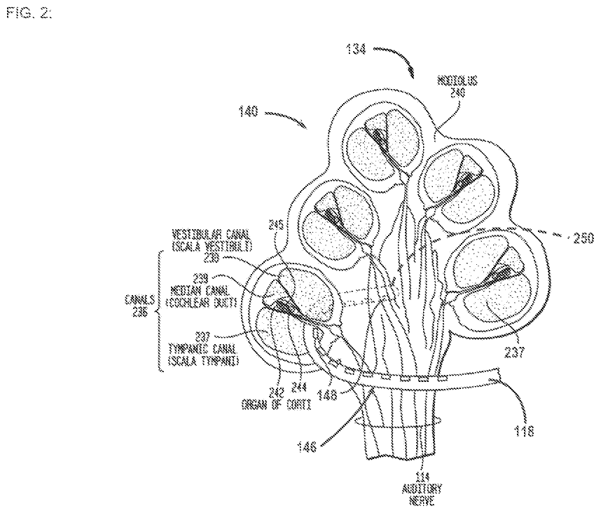

FIG. 2 is cross-sectional view of the cochlea 140 illustrating the stimulating assembly 118 partially implanted therein in accordance with certain implementations described herein. Only a subset of the stimulation elements 148 of the stimulation assembly 118 is shown in FIG. 2. The cochlea 140 is a conical spiral structure that comprises three parallel fluid-filled canals or ducts, collectively and generally referred to herein as canals 236. Canals 236 comprise the tympanic canal 237, also referred to as the scala tympani 237, the vestibular canal 238, also referred to as the scala vestibuli 238, and the median canal 239, also referred to as the scala media 239. The cochlea 140 includes the modiolus 240 which is a conical shaped central region around which the cochlea canals 236 spiral. The modiolus 240 consists of spongy bone in which the cochlea nerve cells, sometimes referred to herein as the spiral ganglion cells, are situated. The cochlea canals 236 generally turn 2.5 times around the modiolus 240.

In normal hearing, sound entering the auricle 110 (see, e.g., FIG. 1) causes pressure changes in the cochlea 140 that travel through the fluid-filled tympanic and vestibular canals 237, 238. The organ of Corti 242, which is situated on the basilar membrane 244 in scala media 239, contains rows of hair cells (not shown) which protrude from its surface. Located above the hair cells is the tectoral membrane 245 which moves in response to pressure variations in the fluid-filled tympanic and vestibular canals 237, 238. Small relative movements of the layers of the tectoral membrane 245 are sufficient to cause the hair cells to move, thereby causing the creation of a voltage pulse or action potential which travels along the associated nerve fibers that connect the hair cells with the auditory nerve 114. The auditory nerve 114 relays the impulses to the auditory areas of the brain (not shown) for processing.

Typically, in cochlear implant recipients, some portion of the cochlea 140 (e.g., the hair cells) is damaged such that the cochlea 140 cannot transduce pressure changes into nerve impulses for relay to the brain. As such, the stimulating elements 148 of the stimulating assembly 118 are used to directly stimulate the cells to create nerve impulses resulting in perception of a received sound (e.g., to evoke a hearing precept).

To insert the intra-cochlear stimulating assembly 118 into the cochlea 140, an opening (facial recess) is created through the recipient's mastoid bone 119 (see, e.g., FIG. 1) to access the recipient's middle ear cavity 106 (see, e.g., FIG. 1). An opening is then created from the middle ear 106 into the cochlea 140 through, for example, the round window 121, oval window 112, the promontory 123, etc. of the cochlea 140. The stimulating assembly 118 is then gently advanced (e.g., pushed) forward into the cochlea 140 until the stimulating assembly 118 achieves the implanted position. As shown in FIGS. 1 and 2, the stimulating assembly 118 follows the helical shape of the cochlea 140. That is, the stimulating assembly 118 spirals around the modiolus 240.

The effectiveness of the stimulation by the stimulation assembly 118 depends, at least in part, on the place along the basilar membrane 244 where the stimulation is delivered. That is, the cochlea 140 has characteristically been referred to as being “tonotopically mapped,” in that regions of the cochlea 140 toward the basal end are more responsive to high frequency signals, while regions of cochlea 140 toward the apical end are more responsive to low frequency signals. These tonotopical properties of the cochlea 140 are exploited in a cochlear implant by delivering stimulation within a predetermined frequency range to a region of the cochlea 140 that is most sensitive to that particular frequency range. However, this stimulation relies on the particular stimulation elements 148 having a final implanted positioned adjacent to a corresponding tonotopic region of the cochlea 140 (e.g., a region of the cochlea 140 that is sensitive to the frequency of sound represented by the stimulation element 148).

To achieve a selected final implanted position, the apical (e.g., distal end/tip) portion 250 of the array 146 is placed at a selected angular position (e.g., angular insertion depth). As used herein, the angular position or angular insertion depth refers to the angular rotation of the apical portion 250 of the array 146 from the cochleostomy 122 (e.g., round window 121) through which the stimulation assembly 118 enters the cochlea 140. As such, the angular position/angular insertion depth may be expressed in terms of how many angular degrees the apical portion 250 has traveled within the cochlea 140 with respect to the cochleostomy 122. For example, an angular insertion depth of one hundred and eighty (180) degrees indicates that the apical portion 250 has traveled around half (½) of the first turn of the cochlea 140. An angular insertion depth of three hundred and sixty (360) degrees indicates that the apical portion 250 has traveled completely around the first turn of the cochlea 140.

In certain implementations, while the stimulation assembly 118 is being implanted (e.g., during a surgical procedure conducted by an operator, such as a medical professional, surgeon, and/or an automated or robotic surgical system), a location and/or an orientation of the array 146 relative to the cochlea 140 (e.g., collectively referred to as the pose of the array 146) is adjusted as the array 146 is advanced and placed into position within the cochlea 140. The goal of the implantation is that the fully-implanted array 146 has an optimal pose in which the array 146 is positioned such that the stimulation elements 148 are adjacent to the corresponding tonotopic regions of the cochlea 140. To achieve the optimal pose, the array 146 is expected to follow a trajectory in the cochlea 140 whereby (i) the stimulation elements 148 are distributed linearly along an axis of the cochlear duct 239, (ii) the array 146 does not make contact with the basilar membrane 244, and (iii) the stimulation elements 148 are in close proximity to the modiolar wall (e.g., if the array 146 is pre-curved) or the stimulation elements 148 are distant from the modiolar wall (e.g., if the array 146 is not pre-curved).

However, one or more these expectations may be violated during insertion of the array 146. For example, the apical portion 250 of the array 146 can become snagged on the wall of the cochlear duct 239, the array 146 can become buckled, folded, and/or overinserted, and/or portions of the cochlea 140 (e.g., scala tympani 237; scala vestibuli 238; cochlear duct 239; organ of Corti 242; basilar membrane 244) can be dislocated, resulting in sub-optimal placement of the array 146. It is desirable to provide the operator with information regarding the pose and/or state of the array 146 (e.g., feedback information provided in real-time during the implantation process). For example, metrics related to the pose of the array 146 (e.g., existence and position of foldover) can be reported continuously, at predetermined intervals, and/or in response to requests by the operator, and alerts regarding events related to insertion (e.g., snagged electrode; other non-optimal conditions) can be provided to the operator, so the operator can take corrective measures.

Electroporation can apply high voltage electrical signals to target cells to increase the cell membrane permeability of the target cells to medicaments and/or other compounds to be introduced within the target cells. For stimulation assemblies 118 configured to apply electroporation signals to target cells, the applied electroporation signals configured to achieve the desired electroporation of the target cells have parameters (e.g., electrical voltages; electrical currents; electrical fields; electrical field gradients; electrical charges) with magnitudes and/or other attributes that are dependent on the placement of the stimulation assembly 118 relative to the target cells. For example, the electroporation voltage magnitudes to achieve a predetermined amount or efficaciousness of electroporation are higher for stimulation elements 148 (e.g., electrodes) spaced farther from the target cells than for stimulation elements 148 spaced closer to the target cells. Thus, electroporation signals configured for use with the stimulation elements 148 at a predetermined location relative to the target cells will not provide the predetermined efficaciousness if the stimulation elements 148 are at an actual location farther away from the target cells than is the predetermined location. In addition, electroporation signals applied using stimulation elements 148 at an actual location spaced away from the predetermined location (e.g., farther from the target cells and closer to non-target cells) can undesirably electroporate the non-target cells.

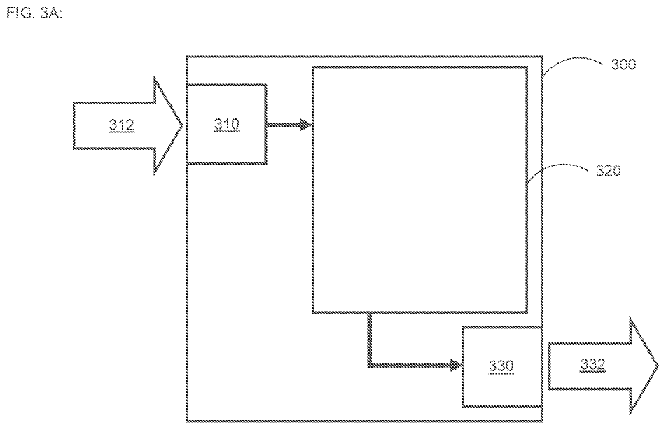

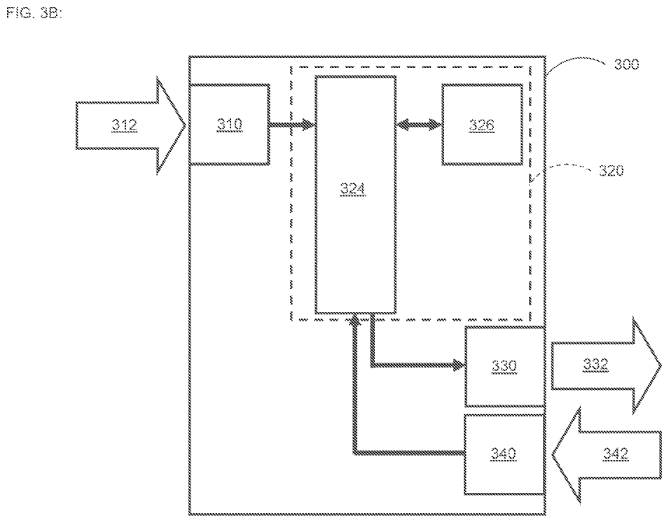

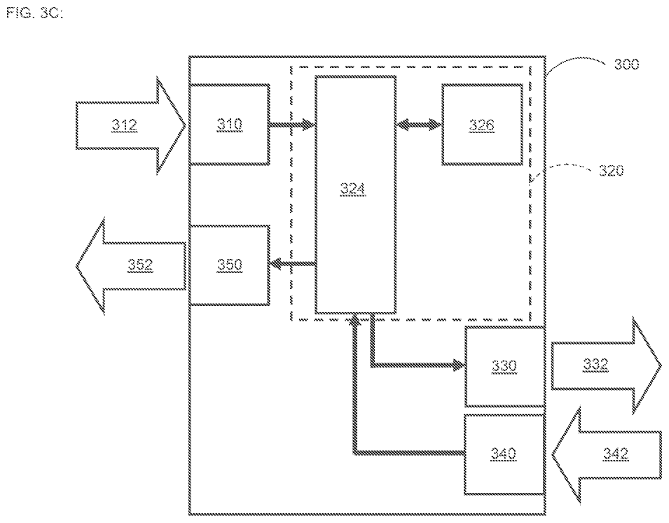

FIGS. 3A-3C schematically illustrate examples of a system 300 in accordance with certain implementations described herein. In certain implementations, the system 300 is configured to use data 312 indicative of a pose (e.g., position and/or orientation) of an implantable device (e.g., medical device) relative to the body portion of the recipient. The implantable device comprises a plurality of electrodes configured to apply electroporation electrical signals to the body portion. For example, the device can comprise a cochlear implant auditory prosthesis 100 having a stimulation assembly 118 with electrodes configured to be implanted into a cochlea 140. At least some of the electrodes can be configured to apply a first set of electrical signals configured to electroporate the target region, to apply a second set of electrical signals configured to stimulate neurons of the body portion (e.g., stimulation signals to invoke a hearing precept), and/or to apply a third set of electrical signals configured to generate data (e.g., transimpedance measurement values; electrical impedance spectroscopy values; electrical field values; electrical potential values; electrical field gradient values; electrical tomography values) indicative of the pose of the device. At least some of the electrodes can be configured to apply electroporation to first cells of the body portion while not damaging second cells of the body portion.

As schematically illustrated by FIG. 3A, in certain implementations, the system 300 comprises at least one data input interface 310 configured to receive data 312 indicative of the pose (e.g., position and/or orientation) of the implantable device relative to the body portion of the recipient. The system 300 further comprises at least one controller 320 in operative communication with the at least one data input interface 310. The at least one controller 320 is configured to access anatomical model data of the body portion. The at least one controller 320 is further configured to generate, in response at least in part to the anatomical model data and the data 312 indicative of the pose of the device, estimated first electrical parameter values at a target region of the body portion and estimated second electrical parameter values at a non-target region of the body portion resulting from electrical signals (e.g., electrical voltages; electrical currents; electrical fields; electrical field gradients; electrical charges) applied to the plurality of electrodes. The at least one controller 320 is further configured to generate an evaluation of whether the estimated first electrical parameter values are sufficient for electroporating the target region and/or the estimated second electrical parameter values are insufficient for damaging the non-target region. The system 300 further comprises at least one output interface 330 in operative communication with the at least one controller 320 and configured to provide information 332 indicative of the evaluation.

In certain implementations, the system 300 comprises the implantable device, while in certain other implementations, the implantable device is separate and external to the system 300. In certain implementations, the system 300 is configured to be controlled, at least in part, by an operator (e.g., medical professional; surgeon; automated or robotic surgical system). For example, the system 300 can comprise at least one computing device (e.g., a desktop computer, a laptop computer, a mobile computing device or accessory; a smartphone; a smart tablet) configured to be utilized by a human operator (e.g., medical professional) and/or the at least one computing device can be a component of an automated or robotic surgical system. As schematically illustrated by FIG. 3B, the system 300 can further comprise at least one operator input interface 340 in operative communication with the at least one controller 320 and configured to be utilized by the human operator and/or automated or robotic surgical system to provide input 342 (e.g., user input; control signals) from the operator to the at least one controller 320.

In certain implementations, the at least one data input interface 310 is configured to receive data prior to, during, and/or after placement (e.g., insertion; implantation) of the device on and/or in the body portion of the recipient. For example, at least some of the data 312 indicative of the pose of the device can be generated during and/or after the placement of the device and the at least one data input interface 310 can be configured to receive at least some of the data 312 during and/or after the placement. In certain implementations, at least some of the data 312 is generated by and received from the plurality of electrodes (e.g., transimpedance measurement values; electrical impedance spectroscopy values; electrical field values; electrical potential values; electrical field gradient values; electrical tomography values). In certain other implementations, at least some of the data 312 is generated by external imaging systems and comprises imaging data (e.g., computed tomography; magnetic resonance; x-ray; fluoroscopy; stereotactic; ultrasound; positron emission tomography) showing the body portion or both the device and the body portion, and is received either from the external imaging system or from an external data source in which the data 312 was stored.

In certain implementations, the at least one data input interface 310 is further configured to receive at least some of the anatomical model data of the body portion prior to, during, and/or after the placement (e.g., from an external imaging system or from an external data source in which the anatomical model data was stored). For example, the anatomical model data can comprise data from imaging (e.g., computed tomography; magnetic resonance; x-ray; fluoroscopy; stereotactic; ultrasound; positron emission tomography) performed on the recipient prior to placement (pre-operatively), during placement, and/or after placement (e.g., post-operatively). In certain other implementations, the anatomical model data can be based on a generic or average anatomy that the recipient is expected to have.

In certain implementations, the at least one controller 320 is configured to generate the evaluation of the sufficiency of the estimated electrical parameter values for electroporating the target region but is not configured to control the plurality of electrodes (see, e.g., FIGS. 3A and 3B). In certain other implementations, the at least one controller 320 is configured to both generate the evaluation and to control the plurality of electrodes of the device (e.g., to generate and apply stimulation signals, to generate at least some of the data 312, and/or to generate and apply electroporation signals). As schematically illustrated by FIG. 3C, the system 300 of certain implementations further comprises at least one control output interface 350 in operative communication with the at least one controller 320 and configured to transmit control signals 352 to the plurality of electrodes, and the electrodes can be responsive to the control signals 352. For example, the control signals 352 can comprise the stimulation signals and/or electroporation signals to be applied by the electrodes (e.g., stimulation elements 148). For another example in which the electrodes are used to generate at least some of the data 312 indicative of the pose of the device (e.g., transimpedance data), the control signals 352 can comprise data acquisition signals (e.g., voltages and/or currents applied by the electrodes) for performing measurements that generate the data 312, the electrodes can be responsive to the control signals 352 by generating at least some of the data 312, and the at least one data input interface 310 can receive the data 312 from the electrodes. In certain such implementations, the at least one controller 320 is configured to generate an estimate of the pose in response at least in part to the data 312 received from the plurality of electrodes and to use the estimated pose in generating the evaluation of the sufficiency of the estimated electrical parameter values for electroporating the target region.

The at least one data input interface 310, the at least one output interface 330, the at least one operator input interface 340, and/or the at least one control output interface 350 can comprise any combination of wired and/or wireless ports, including but not limited to: Universal Serial Bus (USB) ports; Institute of Electrical and Electronics Engineers (IEEE) 1394 ports; PS/2 ports; network ports; Ethernet ports; Bluetooth ports; wireless network interfaces. In certain implementations, the at least one data input interface 310 and the at least one control output interface 350 are integral with one another (e.g., comprising the same ports as one another), while in certain other implementations, the at least one data input interface 310 and the at least one control output interface 350 are separate from one another. In certain implementations, the at least one data input interface 310 and the at least one control output interface 350 are in operative communication with the same electrodes as one another, while in certain other implementations, the at least one data input interface 310 and the at least one control output interface 350 are in operative communication with different electrodes as one another.

In certain implementations, the at least one output interface 330 is configured to provide the information 332 indicative of the evaluation to the operator (e.g., medical professional or surgeon) so that the operator can adjust electrical parameter values applied by the plurality of electrodes for electroporating the target cells in real-time (e.g., during or after implantation of the device). The at least one output interface 330 of certain such implementations is configured to be in operative communication with at least one communication device (e.g., display device; screen; status indicator light; audio device; speaker; vibration motor) configured to communicate information to the operator in real-time. For example, the at least one communication device can be configured to respond to the information 332 by communicating to the operator status signals (e.g., alerts; alarms) indicative of the evaluation, the pose of the device, and/or the operative status of the system 300. In certain other implementations, the at least one output interface 330 is configured to be in operative communication with an automated system (e.g., robotic surgical system) that is configured to respond automatically and in real-time to the information 332 by adjusting electrical parameter values applied by the plurality of electrodes for electroporating the target cells.

In certain implementations, the at least one operator input interface 340 is configured to receive operator input 342 (e.g., commands and/or data) from the operator so that the operator can adjust electrical parameter values applied by the plurality of electrodes for electroporating the target cells in real-time. The at least one operator input interface 340 of certain such implementations is configured to be in operative communication with at least one communication device (e.g., keyboard, computer mouse, touchscreen, switches, buttons) or other device with which a human operator can provide the system 300 with commands and/or data in real-time. In certain other implementations, the at least one operator input interface 340 is configured to be in operative communication with an automated system that is configured to transmit, in real-time, operator input 342 (e.g., commands and/or data) for electroporating the target cells.

In certain implementations, the at least one controller 320 is configured to transmit the control signals 352 to the plurality of electrodes (e.g., stimulation elements 148) automatically (e.g., at a predetermined constant repetition rate; at times determined by the internal logic of the at least one controller 320) during and/or after the implantation of the device. For example, the plurality of electrodes can be activated or triggered to perform data collection automatically upon connection of the system 300 to the plurality of electrodes of the device (e.g., connection of a surgical sound processing unit 126 to a cochlear implant system 100 during implantation). In certain other implementations, the at least one controller 320 is configured to receive triggering signals from the at least one operator input interface 340 intermittently during and/or after the implantation of the device. The at least one controller 320 can be configured to respond to the triggering signals by transmitting the control signals 352 to the plurality of electrodes. In this way, the plurality of electrodes can be selectively activated by the human operator (e.g., by pressing a button of an external device in operative communication with the at least one operator input interface 340) and/or the automated or robotic surgical system.

In certain implementations, as schematically illustrated by FIGS. 3B and 3C, the at least one controller 320 comprises at least one processor 324 and at least one storage device 326 in operative communication with the at least one processor 324. The at least one processor 324 of certain implementations comprises a microprocessor or microcontroller configured to execute instructions (e.g., stored on the at least one storage device 326), to receive data 312 (e.g., via the at least one data input interface 310), to transmit at least some of the received data 312 to the at least one storage device 326 and to access the stored data 312, to generate the evaluation (e.g., based at least in part on the received and/or stored data 312), and to provide information regarding the evaluation (e.g., via the at least one output interface 330; to the at least one storage device 326 to be stored and later retrieved).

The at least one processor 324 can also be configured to generate additional data 312 based at least partially on the data 312 (e.g., a transimpedance gradient vector dataset comprising a plurality of transimpedance gradient vector phase values based on received transimpedance measurement values from the electrodes; an estimated pose of the device) and to store at least a portion of the generated data 312 on the at least one storage device 326. The evaluation generated by the at least one processor 324 can be based, at least in part, on the additional data 312 (e.g., based at least in part on the estimate of the pose).

In certain implementations, the at least one processor 324 is further configured to evaluate and/or filter the received data 312 (e.g., received from the plurality of electrodes). For example, the at least one processor 324 can filter (e.g., in the time domain; using a median filter; using an exponentially weighted moving average filter) data 312 generated by multiple measurements from the electrodes. For other examples, the at least one processor 324 can apply more weighting to more recently generated data 312 (e.g., to selectively apply more weighting to data 312 potentially affected by the presence of the electrode in the body portion), aggregate multiple measurements from an electrode while the electrode is at a location (e.g., time-averaging the data 312 generated by the electrode while at the location), and/or aggregate multiple measurements from multiple electrodes when each of the electrodes is at a predetermined location (e.g., to time-average the data 312 generated by the multiple electrodes while each is at the predetermined location).

The at least one storage device 326 of certain implementations is configured to store at least some of the data 312 (e.g., received from the plurality of electrodes; received from external data sources; generated, evaluated, and/or filtered by the at least one processor 324) and/or the anatomical model data. The at least one storage device 326 can comprise at least one tangible (e.g., non-transitory) computer readable storage medium, examples of which include but are not limited to: read only memory (ROM); random access memory (RAM); magnetic disk storage media; optical storage media; flash memory. The at least one storage device 326 can be encoded with software (e.g., a computer program downloaded as an application) comprising computer executable instructions for instructing a computer system (e.g., measurement logic and/or evaluation logic to be executed by the at least one processor 324). In certain implementations, the at least one processor 324 executes the evaluation logic of the software to generate estimates of the pose of the device, to generate evaluations of the sufficiency of the estimated electrical parameter values for electroporating the target region, and/or to provide the information 332 regarding the estimates and/or the evaluations (e.g., via the at least one output interface 330). In certain implementations, the at least one processor 324 executes the measurement logic of the software to also generate control signals 352 that activate and/or otherwise control the plurality of electrodes.

In certain implementations in which the device 300 is configured to both apply electroporation pulses and to deliver stimulation pulses (e.g., self-administering systems; a cochlear implant or other neurostimulator configured to also perform electroporation), the electroporation pulses can be substantially limited (e.g., constrained) in charge, voltage, current, and/or number of pulses (e.g., due to upper limits resulting from safety concerns) as compared to devices that are dedicated to electroporation (e.g., separate from a neurostimulator). For example, the total unidirectional charge that can be applied by capacitively coupled electrodes of a self-administering system can be substantially constrained due to the fact that the capacitors charge during the delivery of electroporation, thereby preventing further current flow as more pulses are applied. For another example, the maximum voltage applied between any two electrodes of an electrode pair can be constrained since the implant electronics can be damaged by high voltages. In certain such implementations, the evaluation of the sufficiency of the electrical parameter values generated by the at least one controller 320 can be used to determine an optimal placement of the electrodes to perform the electroporation despite the constraints of such stimulation and electroporation systems.

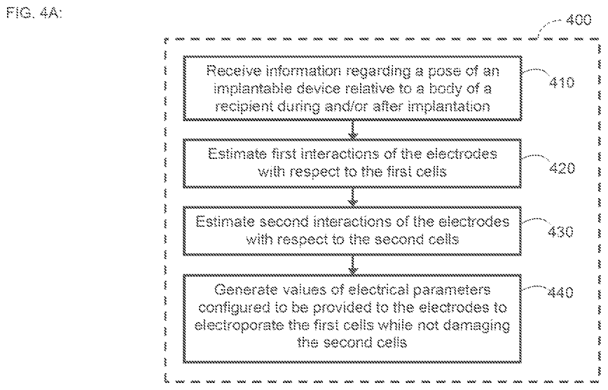

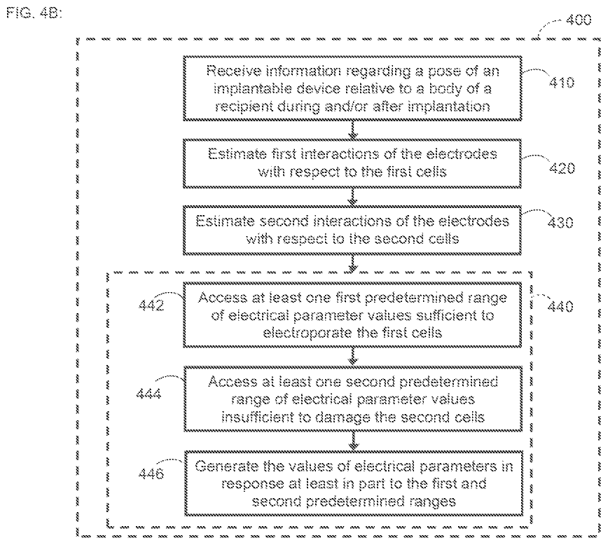

FIGS. 4A and 4B are flow diagram of examples of a method 400 in accordance with certain implementations described herein. While the method 400 is described by referring to some of the structures of the example system 300 of FIGS. 3A-3C, other systems with other configurations of components can also be used to perform the method 400 in accordance with certain implementations described herein. In certain implementations, the method 400 can be performed using a device comprising electrodes configured for multiple use (e.g., dual use). For example, at least some of the electrodes can be configured to provide stimulation signals to cells of the body portion and electroporation signals to cells of the body portion (e.g., either the same or different cells that receive the stimulation signals). At least some of the electrodes that provide the stimulation signals and/or the electroporation signals can also be configured to generate information indicative of the pose of the device. In certain implementations, a non-transitory computer readable storage medium has stored thereon a computer program that instructs a computer system to perform the method 400 (e.g., to provide real-time information regarding electroporation to be applied by electrodes of a device to first cells of a body portion of a recipient during and/or after implantation of the device on and/or within the body portion).

In an operational block 410, the method 400 comprises receiving information (e.g., data 312) regarding a pose (e.g., a position and/or an orientation) of the implantable device relative to the body portion of the recipient during and/or after implantation of at least a portion of the device on and/or into the body portion. In certain implementations, the portion of the device comprises a plurality of electrodes configured to apply electroporation to first cells of the body portion while not damaging second cells of the body portion, and the received information can comprise information regarding the shape of the tissue in which the device is implanted and/or the proximity of the electrodes to the first and/or second cells. For example, the first cells and the second cells can be located within a cochlea 140 of a recipient (e.g., the first cells comprising mesenchymal cells lining a scala tympani and/or a basilar membrane of the cochlea 140; the second cells comprising hair cells of the cochlea 140) and the received information can be indicative of the relative position and/or orientation of the electrodes relative to the first cells and the second cells.

In certain implementations, at least some of the received information is generated by and received from the plurality of electrodes of the device during and/or after the implantation of the portion of the device (e.g., transimpedance measurement values; electrical impedance spectroscopy values; electrical field values; electrical potential values; electrical field gradient values; electrical tomography values). For example, at least some of the received information can be generated by an operator of an insertion system being used to implant the portion of the device or by an automated implantation system performing the implantation.

For certain other implementations, at least some of the received information is imaging data showing the body portion or both the device and the body portion (e.g., computed tomography imaging data; magnetic resonance imaging data; x-ray imaging data; fluoroscopy imaging data; stereotactic imaging data; ultrasound imaging data; positron emission tomography imaging data) generated before, during, and/or after the implantation of the device and is received either from the external imaging system that generated the imaging data or from an external data source in which the imaging data was stored. In certain implementations, the received information comprises anatomical model data of the body portion prior to, during, and/or after the placement (e.g., from an external imaging system or from an external data source in which the anatomical model data was stored). For example, the anatomical model data can comprise data from imaging (e.g., computed tomography; magnetic resonance; x-ray; fluoroscopy; stereotactic; ultrasound; positron emission tomography) performed on the recipient prior to placement (pre-operatively), during placement, and/or after placement (e.g., post-operatively). In certain other implementations, the anatomical model data can be based on a generic or average anatomy that the recipient is expected to have.

At least some of the electrodes can be configured to apply electroporation to first cells of the body portion while not damaging second cells of the body portion. For example, for electroporation of cells of the cochlea 140, the first cells can comprise mesenchymal cells lining a scala tympani and/or a basilar membrane of the cochlea 140 and the second cells can comprise hair cells of the cochlea 140. For another example, for electroporation of tumor cells, the first cells can comprise target tumor cells and the second cells can comprise healthy, non-tumor cells. For yet another example, for electroporation of an organ (e.g., brain), the first cells can comprise the target cells of the organ (e.g., neurons to be damaged via electroporation) and the second cells can comprise the non-target cells of the organ (e.g., neurons not to be damaged via electroporation).

In an operational block 420, the method 400 further comprises estimating, in response at least in part to the information, first interactions (e.g., electrical interactions) of the electrodes with respect to the first cells. For example, for a stimulating assembly 118 comprising an array 146 of electrodes configured to deliver electrical parameters (e.g., electrical voltages; electrical currents; electrical fields; electrical field gradients; electrical charges) to the cochlea 140, at least some of the electrodes can be used to generate data (e.g., transimpedance measurement values; electrical impedance spectroscopy values; electrical field values; electrical potential values; electrical field gradient values; electrical tomography values) regarding the pose of the stimulating assembly 118 relative to the cochlea 140 and the first electrical interactions of the electrodes of the stimulating assembly 118 with respect to the first cells (e.g., mesenchymal cells lining a scala tympani and/or a basilar membrane of the cochlea) can be estimated (e.g., measured). For another example, the first electrical interactions of the electrodes of the stimulating assembly 118 from the first cells can be estimated (e.g., measured) from imaging data (e.g., computed tomography data) that includes the location (e.g., distance) of the stimulating assembly 118 relative to the cochlea 140.

In an operational block 430, the method 400 further comprises estimating, in response at least in part to the information, second interactions (e.g., electrical interactions) of the electrodes with respect to the second cells. For example, for a stimulating assembly 118 comprising an array 146 of electrodes configured to deliver electrical signals (e.g., electrical voltages; electrical currents; electrical fields; electrical field gradients; electrical charges) to the cochlea 140, at least some of the electrodes can be used to generate data (e.g., transimpedance measurement values; electrical impedance spectroscopy values; electrical field values; electrical potential values; electrical field gradient values; electrical tomography values) regarding the pose of the stimulating assembly 118 relative to the cochlea 140 (e.g., the same data that is used to estimate the first electrical interactions) and the second electrical interactions of the electrodes of the stimulating assembly 118 from the second cells (e.g., hair cells of the cochlea) can be estimated (e.g., measured). For another example, the second electrical interactions of the electrodes of the stimulating assembly 118 from the second cells can be estimated (e.g., measured) from imaging data (e.g., computed tomography data) that includes the location (e.g., distance) of the stimulating assembly 118 relative to the cochlea 140 (e.g., the same imaging data that is used to estimate the first electrical interactions). In an operational block 440, the method 400 further comprises generating, in response at least in part to the estimated first interactions and the estimated second interactions, values of electrical parameters (e.g., electrical voltages; electrical currents; electrical fields; electrical field gradients; electrical charges) configured to be provided to the plurality of electrodes to electroporate the first cells while not damaging the second cells. For example, as shown in FIG. 4B, in an operational block 442, generating the values of electrical parameters can comprise accessing (e.g., from the at least one storage device 326 and/or from operator input 342) at least one first predetermined range of electrical parameter values sufficient to electroporate the first cells (e.g., minimum electrical parameter values that when applied to the electrodes would electroporate the first cells). In an operational block 444, generating the values of electrical parameters can further comprise accessing (e.g., from the at least one storage device 326 and/or from operator input 342) at least one second predetermined range of electrical parameter values insufficient to damage the second cells (e.g., maximum electrical parameter values that when applied to the electrodes would not electroporate or otherwise damage the second cells). In an operational block 446, generating the values of electrical parameters can be in response at least in part to the at least one first predetermined range of electrical parameter values and the at least one second predetermined range of electrical parameter values (e.g., selecting electrical parameter values between the minimum electrical parameter values that would electroporate the first cells and the maximum electrical parameter values that would not damage the second cells).

Electrical interactions of the electrodes with respect to the first and second cells are at least partially dependent on the location (e.g., distance) of the electrodes from the first and second cells, as well as electrical field shapes generated by the electrodes and the electrical properties of the tissue in which the electrodes are implanted. In certain implementations, the received information is used to estimate the locations (e.g., distances) of the electrodes from the first and/or second cells, and these estimated locations are used to generate the values of electrical parameters to be used for electroporating the first cells while not damaging (e.g., not electroporating) the second cells.

The first and second predetermined ranges of electrical parameter values can be accessed either in real-time (e.g., during electroporation and/or implantation) or in advance of the implantation surgery. The source of the first and second predetermined ranges can be prior in vitro experiments (see, e.g., J. L. Pinyon et al., “Close-Field Electroporation Gene Delivery Using the Cochlear Implant Electrode Array Enhances the Bionic Ear,” Sci. Translational Med., Vo. 6, 233ra54 (2014); J. L. Pinyon et al., “Neurotrophin gene augmentation by electrotransfer to improve cochlear implant hearing outcomes,” Hearing Research, Vol. 380, pp. 137-149 (2019)) and/or computer simulation models of the body portion in which electroporation is to be performed (see, e.g., J. H. M. Frijns et al., “Spatial selectivity in a rotationally symmetric model of the electrically stimulated cochlea,” Hearing Research, Vol. 95, pp. 33-48 (1996); P. Wong et al., “Development and Validation of a High-Fidelity Finite-Element Model of Monopolar Stimulation in the Implanted Guinea Pig Cochlea,” IEEE Trans. Biomed. Eng., Vol. 63, No. 1, pp. 188-198 (2016); A. A. Abed et al., “Computational Simulation Expands Understanding of Electrotransfer-Based Gene Augmentation for Enhancement of Neural Interfaces,” Frontiers in Neuroscience, Vol. 13, Article 691 (2019)).

In certain implementations, the method 400 further comprises evaluating a suitability for electroporation of the position and/or orientation of the device relative to the body portion and communicating the evaluation in real-time during implantation of the device to either an operator of an electroporation system or to an automated electroporation system. For example, the method 400 can comprise generating a feedback signal indicative of the pose of the device and/or the suitability for electroporation of the pose of the device. The feedback signal can be provided in real-time (e.g., during the implantation; during the electroporation) to a human operator or to an automated system performing the implantation and/or electroporation. In certain implementations in which the feedback signal is indicative of the evaluation of the suitability for electroporation, the feedback signal can be used for closed loop feedback control to optimize the pose of the device for electroporating the first cells while not damaging the second cells. In certain implementations in which the device also provides stimulation signals to the body portion, the pose of the device can also be optimized for efficient stimulation. For example, the system 300 and/or method 400 can be used to provide closed loop feedback control to achieve optimal placement of a gene delivery array (GDA) (e.g., the GDA electrodes close to the mesenchymal cells lining the scala tympani of the cochlea 140). The information regarding the pose of the GDA (e.g., data regarding the angular or linear depth of insertion of the GDA within the cochlea 140) can be used to estimate the first and second interactions of the GDA electrodes with respect to the first and second cells, respectively, and the electroporation parameter values to deliver the gene delivery medicament to the first cells and/or an evaluation of the sufficiency of the electroporation parameter values for electroporating the first cells while not electroporating the second cells. The information 332 can be provided to the operator as a feedback signal to use while adjusting the pose of the GDA (e.g., by changing the position of the GDA through surgical manipulation or automatic control) to achieve optimal placement of the GDA. For example, the feedback signal can be in the form of an indicator signal (e.g., red, yellow, or green indicator) indicative of the likely effectiveness of electroporation using the GDA in its current pose.

Although commonly used terms are used to describe the systems and methods of certain implementations for ease of understanding, these terms are used herein to have their broadest reasonable interpretations. Although various aspects of the disclosure are described with regard to illustrative examples and implementations, the disclosed examples and implementations should not be construed as limiting. Conditional language, such as, among others, “can,” “could,” “might,” or “may,” unless specifically stated otherwise, or otherwise understood within the context as used, is generally intended to convey that certain implementations include, while other implementations do not include, certain features, elements and/or steps. Thus, such conditional language is not generally intended to imply that features, elements and/or steps are in any way required for one or more implementations or that one or more implementations necessarily include logic for deciding, with or without user input or prompting, whether these features, elements and/or steps are included or are to be performed in any particular implementation. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced.

It is to be appreciated that the implementations disclosed herein are not mutually exclusive and may be combined with one another in various arrangements. In addition, although the disclosed methods and apparatuses have largely been described in the context of conventional cochlear implants, various implementations described herein can be incorporated in a variety of other suitable devices, methods, and contexts. More generally, as can be appreciated, certain implementations described herein can be used in a variety of implantable medical device contexts that can benefit from having at least a portion of the received power available for use by the implanted device during time periods in which the at least one power storage device of the implanted device unable to provide electrical power for operation of the implantable medical device.

Language of degree, as used herein, such as the terms “approximately,” “about,” “generally,” and “substantially,” represent a value, amount, or characteristic close to the stated value, amount, or characteristic that still performs a desired function or achieves a desired result. For example, the terms “approximately,” “about,” “generally,” and “substantially” may refer to an amount that is within ±10% of, within ±5% of, within ±2% of, within ±1% of, or within ±0.1% of the stated amount. As another example, the terms “generally parallel” and “substantially parallel” refer to a value, amount, or characteristic that departs from exactly parallel by ±10 degrees, by ±5 degrees, by ±2 degrees, by ±1 degree, or by ±0.1 degree, and the terms “generally perpendicular” and “substantially perpendicular” refer to a value, amount, or characteristic that departs from exactly perpendicular by ±10 degrees, by ±5 degrees, by ±2 degrees, by ±1 degree, or by ±0.1 degree. The ranges disclosed herein also encompass any and all overlap, sub-ranges, and combinations thereof. Language such as “up to,” “at least,” “greater than,” less than,” “between,” and the like includes the number recited. As used herein, the meaning of “a,” “an,” and “said” includes plural reference unless the context clearly dictates otherwise. Also, as used in the description herein, the meaning of “in” includes “into” and “on,” unless the context clearly dictates otherwise.

While the methods and systems are discussed herein in terms of elements labeled by ordinal adjectives (e.g., first, second, etc.), the ordinal adjective are used merely as labels to distinguish one element from another (e.g., one signal from another or one circuit from one another), and the ordinal adjective is not used to denote an order of these elements or of their use.

The invention described and claimed herein is not to be limited in scope by the specific example implementations herein disclosed, since these implementations are intended as illustrations, and not limitations, of several aspects of the invention. Any equivalent implementations are intended to be within the scope of this invention. Indeed, various modifications of the invention in form and detail, in addition to those shown and described herein, will become apparent to those skilled in the art from the foregoing description. Such modifications are also intended to fall within the scope of the claims. The breadth and scope of the invention should not be limited by any of the example implementations disclosed herein, but should be defined only in accordance with the claims and their equivalents.

1. A method comprising:
receiving information regarding a position and/or an orientation of an implantable device relative to a body portion of a recipient during and/or after implantation of at least a portion of the device on and/or into the body portion, the device comprising a plurality of electrodes configured to apply electroporation to first cells of the body portion while not damaging second cells of the body portion;
estimating, in response at least in part to the information, first interactions of the electrodes with respect to the first cells;
estimating, in response at least in part to the information, second interactions of the electrodes with respect to the second cells; and
generating, in response at least in part to the estimated first interactions and the estimated second interactions, values of electrical parameters configured to be provided to the plurality of electrodes to electroporate to the first cells while not damaging the second cells.

2. The method of claim 1, wherein the first cells and the second cells are located within a cochlea of the recipient.

3. The method of claim 2, wherein the first cells comprise mesenchymal cells lining a scala tympani and/or a basilar membrane of the cochlea and the second cells comprise hair cells of the cochlea.

4. The method of claim 1, wherein the information comprises one or more values selected from the group consisting of: transimpedance measurement values; electrical impedance spectroscopy values; electrical field values; electrical potential values; electrical field gradient values; electrical tomography values.

5. The method of claim 1, wherein the information comprises imaging data comprising one or more of the group consisting of: computed tomography imaging data; magnetic resonance imaging data; x-ray imaging data; stereotactic imaging data; ultrasound data; positron emission tomography data.

6. The method of claim 5, wherein at least some of the imaging data comprises pre-operative imaging data.

7. The method of claim 1, wherein the information further comprises electrocochleography data or fluoroscopy data.

8. The method of claim 1, wherein at least some of the information is generated by the device during implantation of the portion of the device on and/or into the body portion.

9. The method of claim 8, the method further comprising generating a feedback signal indicative of the position and/or orientation of the device during the implantation and providing, in real-time, the feedback signal to an operator of an insertion system being used to implant the device and/or to an automated implantation system performing the implantation.

10. The method of claim 1, further comprising evaluating a suitability for electroporation of the position and/or orientation of the device relative to the body portion and communicating the evaluation, in real-time during implantation of the device on and/or into the body portion, to either an operator of an electroporation system or to an automated electroporation system.

11. The method of claim 1, wherein generating the values of electrical parameters comprises:
accessing at least one first predetermined range of electrical parameter values sufficient to electroporate the first cells;
accessing at least one second predetermined range of electrical parameter values insufficient to damage the second cells; and
generating the values of electrical parameters in response at least in part to the at least one first predetermined range of electrical parameter values and the at least one second predetermined range of electrical parameter values.

12. A system comprising:
at least one data input interface configured to receive data indicative of a pose of a medical device relative to a body portion of a recipient, the medical device comprising a plurality of electrodes configured to apply electroporation signals to the body portion;
at least one controller in operative communication with the at least one data input interface, the at least one controller configured to:
access anatomical model data of the body portion;
in response at least in part to the anatomical model data and the data indicative of the pose of the medical device, generate estimated first electrical parameter values at a target region of the body portion and estimated second electrical parameter values at a non-target region of the body portion resulting from electrical signals applied to the plurality of electrodes; and
generate an evaluation of whether the estimated first electrical parameter values are sufficient for electroporating the target region and/or the estimated second electrical parameter values are insufficient for damaging the non-target region; and
at least one output interface in operative communication with the at least one controller, the at least one output interface configured to provide information indicative of the evaluation.

13. The system of claim 12, wherein the at least one data input interface is configured to receive the data during and/or after implantation of the medical device on and/or in the body portion.

14. The system of claim 12, wherein the body portion comprises a cochlea of the recipient and the plurality of electrodes comprises electrodes of a stimulation assembly of a cochlear implant auditory prosthesis configured to be inserted at least partially into the cochlea.

15. The system of claim 12, wherein the plurality of electrodes are configured to apply a first set of electrical signals configured to electroporate the target region and to apply a second set of electrical signals configured to stimulate neurons of the body portion.

16. The system of claim 12, wherein the at least one controller comprises at least one processor and at least one storage device in operative communication with the at least one processor, the at least one storage device configured to store the anatomical model data and the data indicative of the pose of the medical device.

17. The system of claim 12, further comprising at least one control output interface in operative communication with the at least one controller, the at least one control output interface configured to transmit control signals to the plurality of electrodes, the plurality of electrodes responsive to the control signals by generating at least some of the data indicative of the pose of the medical device.

18. The system of claim 17, wherein the at least one controller is configured to transmit the control signals to the plurality of electrodes automatically during implantation of the medical device.

19. The system of claim 17, further comprising at least one operator input interface in operative communication with the at least one controller, the at least one controller configured to receive triggering signals from the at least one operator input interface intermittently during implantation of the medical device, wherein the at least one controller is configured to respond to the triggering signals by transmitting the control signals to the plurality of electrodes.

20. The system of claim 19, wherein the at least one operator input interface and the at least one output interface are configured to be in operative communication with a computing device configured to be utilized by a medical professional.

21. The system of claim 12, wherein the at least one output interface is configured to be in operative communication with an automated system configured to respond automatically and in real-time to the information by adjusting electrical parameter values applied by the plurality of electrodes for electroporating the target cells.

22. The system of claim 12, wherein the at least one output interface is configured to be in operative communication with at least one communication device configured to respond to the information by communicating status signals indicative of the evaluation.

23. A non-transitory computer readable storage medium having stored thereon a computer program that instructs a computer system to provide real-time information regarding electroporation to be applied by electrodes of a device to first cells of a body portion of a recipient during and/or after implantation of the device on and/or within the body portion by at least:
receiving information regarding a pose of the device relative to the body portion during and/or after implantation of the device;
estimating, in response at least in part to the information, first interactions of the electrodes with respect to the first cells;
estimating, in response at least in part to the information, second interactions of the electrodes with respect to second cells different from the first cells; and
generating, in response at least in part to the estimated first interactions and the estimated second interactions, values of electrical parameters configured to be provided to the plurality of electrodes to electroporate to the first cells while not damaging the second cells.
